(19) United States
(12) Patent Application Publication
(10) Pub. No.: US 2006/0149051 A1
(43) Pub. Date: Jul. 6, 2006

(54) Method of preparing an acylimidazolium-type reagent

(76) Inventors: Thierry Schlama, Dardilly (FR); Franco Manfre, Ecully (FR)

Correspondence Address: Jean-Louis Seugnet;Rhodia Inc.-Legal Department, CN7500, 8 Cedar Brook Drive, Cranbury, NJ, 08512-7500, US

(21) Appl. No.: 10/542,249

(22) PCT Filed: Jan. 14, 2004
(86) PCT No.: PCT/FR04/00059

§ 371 Date: Jan. 20, 2006

(30) Foreign Application Priority Data
Jan. 15, 2003 (FR) 03 00390

(51) Int. Cl. C07H 21/04; C07D 233/91

(52) U.S. Cl. 536/25.3; 548/328.1

(57) ABSTRACT

The invention relates to a method of preparing an acylimidazolium-type reagent. More specifically, the invention relates to a salt of N-(benzyloxycarbonyl)-N′-methylimidazolium. The inventive method of preparing an acylimidazolium-type reagent is characterised in that it is obtained by: (i) reacting a reagent comprising a —COX group having formula (II) and an imidazole reagent having formula (III); and (ii) subsequently, adding a strong acid HY to the product thus obtained, said acid having a pKa of less than 1, which produces the desired reagent.

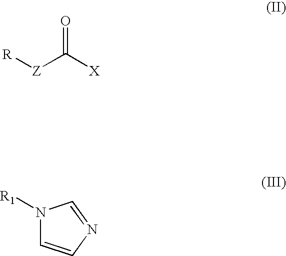

The present invention relates to a method of preparing an acylimidazolium-type reagent.

The invention relates more particularly to an N-(benzyloxycarbonyl)-N′-methylimidazolium salt.

In a multistep synthesis method developed in the field of organic synthesis, it is common practice to protect certain functional groups, and in particular amino groups.

Mention may in particular be made of the case where the exo amino group of nucleic acid bases, such as, for example, adenine, is protected.

A reagent that is particularly useful because it applies to relatively unreactive amino groups consists of the Rapoport reagent, which can be represented by the following formula: 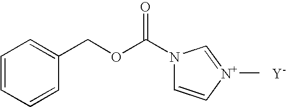
in said formula, Y is an anion and represents, for example, a tetrafluoroborate or trifluoro-methanesulfonate anion.

Bruce E. Watkins and Henry Rapoport described (J. Org. Chem. 1982, 47, 4471-4477) a method for obtaining N-(benzyloxycarbonyl)-N′-ethylimidazolium tetrafluoroborate which comprises two steps. The first consists in reacting imidazole and benzyl chloroformate, to give an imidazolide which is separated by crystallization and then subsequently reacted with triethyloxonium tetrafluoroborate so as to obtain, after crystallization, N-(benzyloxycarbonyl)-N′-ethylimidazolium tetrafluoroborate, which is then used as a reagent to protect an amino group.

The drawbacks of the method described lie in the fact that it comprises two steps with separation of the intermediate product. Said method involves an alkylation step which makes use of triethyloxonium tetrafluoroborate, which is an expensive reagent that is commercially available in solution diluted in dichloromethane and which, moreover, exhibits high toxicity.

The aim of the present invention is to provide a method that can be more readily implemented on an industrial scale and that does not have the abovementioned drawbacks.

A method has now been found, and this is the subject of the present invention, which is a method of preparing an acylimidazolium-type reagent of formula (I): 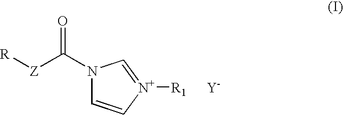
in said formula:
R₁ represents an alkyl or phenyl group,
R represents an alkyl, alkenyl, cycloalkyl, aryl or arylalkyl group,
Z represents a valency bond, an oxygen atom or an NR₂ group; R₂ having the same meaning as R,
Y is an anion originating from an acid whose pKa is less than 1,
   characterized in that it is obtained by reacting:
a reagent comprising a group —COX and corresponding to formula (II): 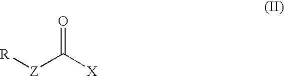
   in said formula:
R and Z have the meaning given above,
X represents a bromine or chlorine atom,
and an imidazole reagent of formula (III): 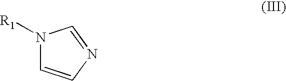
   in said formula:
R₁ has the meaning given above,
and then by adding, to the product obtained, a strong acid HY having a pKa of less than 1, which gives the reagent of formula (I), which is recovered.

In the context of the invention, the term “alkyl” is intended to mean a linear or branched hydrocarbon-based chain having from 1 to 12 carbon atoms, and preferably from 1 to 4 carbon atoms.

Examples of preferred alkyl groups are in particular methyl, ethyl, propyl, isopropyl, butyl, isobutyl or t-butyl.

The term “alkenyl” is intended to mean a linear or branched hydrocarbon-based group having from 2 to 12 carbon atoms, comprising one or more double bonds, preferably 1 or 2 double bonds. The allyl group is a preferred example.

The term “cycloalkyl” is intended to mean a monocyclic, cyclic hydrocarbon-based group comprising from 3 to 8 carbon atoms, preferably a cyclopentyl or cyclohexyl group.

The term “aryl” is intended to mean a monocyclic or polycyclic aromatic group, preferably monocyclic or bicyclic, comprising from 6 to 12 carbon atoms, preferably phenyl or naphthyl.

The phenyl group is preferred.

The term “arylalkyl” is intended to mean a linear or branched hydrocarbon-based group carrying a monocyclic aromatic ring and comprising from 7 to 12 carbon atoms, preferably benzyl.

The starting reagent of formula (II) comprises a group —CO—X.

It is found in a certain number of compounds that can be used in the method of the invention.

A first class is those of carboxylic acid chloride or bromide type, of formula (II), in which Z represents a valency bond.

The preferred compounds are those of formula (II) in which R represents a linear or branched alkyl group having from 1 to 4 carbon atoms, preferably a methyl or ethyl group.

As a preferred reagent, mention may be made of acetyl chloride.

A second family of compounds consists of the compounds of chloroformate or bromoformate type.

Those of formula (II) in which Z represents an oxygen atom and R preferably represents a linear or branched alkyl group having from 1 to 4 carbon atoms, or a benzyl group, are preferably chosen.

The preferred compounds are alkyl or benzyl chloroformate or bromoformate.

Another class of compounds consists of those of formula (II) in which Z represents a group NR₂.

The compounds envisaged are of the carbamoyl chloride or bromide type.

Those of formula (II) in which R and R₂ are identical and preferably represent a linear or branched alkyl group having from 1 to 4 carbon atoms are preferably chosen.

As a preferred reagent, mention may be made of dimethylcarbamoyl chloride.

As regards the imidazole reagent, it is a nitrogenous heterocyclic compound which corresponds to formula (III) and which carries, on the ring, a group R₁ which is a linear or branched alkyl group having from 1 to 12 carbon atoms or a phenyl group. It should be noted that these preferred meanings are indicated, but are not limiting in nature. However, given that this group is considered to be a leaving group when the reagent of formula (I) is used as a protective group, in particular for protecting amino groups, it is advantageous, from an economical point of view, for it to be simple in nature and to represent more particularly a linear or branched alkyl group having from 1 to 4 carbon atoms, preferably a methyl group.

The compounds corresponding to formula (III) are known products. Some are commercially available and others can be readily prepared by those skilled in the art. Thus, those of formula (III) in which R₁ represents an alkyl group can be prepared by the alkylation of imidazole with a bromoalkane (Journal of Organic Chemistry 1999, 64 (3), p. 807-818). A compound of formula (III) in which R₁ represents a phenyl group is obtained by means of a coupling reaction between the imidazole and a halobenzene compound in the presence of a copper-based, palladium-based or nickel-based catalyst (Journal of American Chemical Society 2001, 123 (31), p. 7727-7729).

A preferred reagent is N-methylimidazole.

The third reagent involved in the method of the invention is a strong acid HY, the characteristic of which is to have a pKa in water of less than 1.0.

The pKa is defined as the ionic dissociation constant of the acid/base couple when water is used as solvent.

The anion Y⁻ should be non-nucleophilic. More specifically, it should not react in solution with the compound obtained, i.e. the acylimidazolium.

More specific examples that may be mentioned are: BF₄⁻, PF₆⁻, SO₃F⁻ and CF₃SO₃⁻.

Trifluoromethanesulfonic acid, commonly called “triflic acid” is preferably chosen.

Use is made of a concentrated, preferably pure, strong acid in order to minimize the introduction of water.

In accordance with the method of the invention, the three reagents are reacted without the intermediate product being isolated.

Moreover, use is made of reagents that are in liquid form and that can therefore be conveyed by means of pumps. Consequently, they are more readily used industrially compared with a solid form.

First, the reagent comprising a group —COX of formula (II) and the imidazole-type reagent of formula (III) are reacted.

The amount of the reagents used is such that the ratio of the number of moles of reagent of formula (III) to the number of moles of reagent of formula (II) is advantageously chosen between 1 and 1.2, and preferably in the region of 1.

A preferred embodiment of the invention consists in carrying out the reaction in an organic solvent.

There are several imperatives that determine the choice of the solvent.

It should be inert under the conditions of the invention, in particular with respect to the strong acid.

Use is preferably made of an aprotic and relatively nonpolar organic solvent.

As examples of solvents that are suitable for the present invention, mention may in particular be made of halogenated or nonhalogenated, aliphatic, cycloaliphatic or aromatic hydrocarbons.

By way of examples of aliphatic hydrocarbons, mention may more particularly be made of paraffins such as, in particular, hexane, cyclohexane, methylcyclohexane, petroleum cuts of the petroleum ether type; aromatic hydrocarbons such as, in particular, benzene, toluene, xylenes, cumene, petroleum cuts consisting of a mixture of alkylbenzenes, in particular the Solvesso® type cuts.

As more specific examples of organic solvents, mention may be made of halogenated aliphatic hydrocarbons, and more particularly n-chlorobutane, dichloromethane, 1,2-dichloroethane; halogenated aromatic hydrocarbons, and more particularly mono- or dichlorobenzene.

A mixture of organic solvents can also be used.

The preferred solvents are: dichloromethane or toluene.

The amount of organic solvent used is such that the concentration of the reagents of formula (II) and (III) in the solvent is between 5% and 30% by weight.

The reaction is carried out at a temperature that is advantageously between 0° C. and 30° C., preferably at ambient temperature. The term “ambient temperature” is most commonly intended to mean a temperature of between 15° C. and 25° C.

Generally, the reaction is carried out at atmospheric pressure, but lower or higher pressure may also be suitable.

According to a preferred variant of the method of the invention, the method of the invention is carried out under a controlled atmosphere of inert gases. A rare gas, preferably argon, atmosphere can be established, but it is more economical to make use of nitrogen.

According to another preferred variant of the method of the invention, the reaction is carried out with stirring and in dry conditions.

From a practical point of view, the method can be carried out in a batch or continuous manner.

According to a preferred embodiment of the method of the invention, the solvent is first introduced, and then the reagent of formula (II).

Next, the reagent of formula (III) is added, preferably gradually (continuously or in fractions).

A product forms in suspension, which product corresponds to formula (IV): 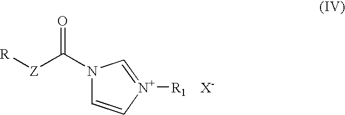
in said formula, R, R₁, X and Z have the meanings given for formulae (II) and (III).

This product is not isolated, and is kept in suspension by stirring.

The strong acid, preferably triflic acid, is added.

The amount of acid added is such that the ratio of the number of H⁺ ions to the number of moles of product of formula (IV), the product obtained subsequent to the reaction of the reagents (II) and (III), ranges between 0.9 and 1.5, preferably between 1 and 1.1.

According to a preferred embodiment, the acid is added to the reaction medium gradually.

A completely clear homogeneous solution comprising the reagent of formula (I) is obtained.

This reagent, which provides a protective group of R—Z—CO— type that can be used to block functional groups, preferably amino groups, can therefore be used in the form of the solution obtained above. It is also possible to use it in solid form, obtained after elimination of the reaction solvent by evaporation.

Said reagent is advantageously used for protecting the amino or substituted amino groups present in any type of molecule.

It is in particular suitable for protecting the functional groups present in primary and secondary amines.

By way of illustration, mention may be made of the primary and secondary amines that can be represented by the following formula: 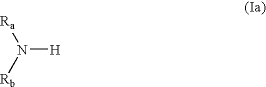
in said formula (Ia):
Rₐ and R_{b} represent, independently of one another, a hydrogen atom or a hydrocarbon-based group having from 1 to 20 carbon atoms, which can be a saturated or unsaturated, linear or branched, acyclic aliphatic group; a monocyclic or polycyclic, saturated, unsaturated or aromatic, carbocyclic or heterocyclic group; a series of the abovementioned groups,
Rₐ and R_{b} can be attached so as to constitute, with the carbon atoms which carry them, a monocyclic or polycyclic, saturated, unsaturated or aromatic, heterocyclic group having from 3 to 20 atoms,
at most one of the groups Rₐ and R_{b} represents a hydrogen atom.

In formula (Ia), the various symbols can more particularly have the meaning given hereinafter.

Thus, Rₐ and R_{b} can represent, independently of one another, a saturated or unsaturated, linear or branched, acyclic aliphatic group.

More specifically, Rₐ and R_{b} preferably represent a saturated, linear or branched, acyclic aliphatic, preferably C₁ to C₁₂, and even more preferably C₁ to C₄, group.

The invention does not exclude the presence of an unsaturation on the hydrocarbon-based chain, such as one or more double bonds which may or may not be conjugated.

The hydrocarbon-based chain can be optionally interrupted with a hetero atom (for example, oxygen, sulfur, nitrogen or phosphorous) or with a functional group, provided that the latter does not react, and a group such as in particular —CO— may in particular be mentioned.

The hydrocarbon-based chain can optionally carry one or more substituents (for example, halogen, carboxylic, ester, amino or alkyl and/or arylphosphine) provided that they do not interfere.

The saturated or unsaturated, linear or branched, acyclic aliphatic group can optionally carry a cyclic substituent. The term “ring” is intended to mean a saturated, unsaturated or aromatic, carbocyclic or heterocyclic ring.

The acyclic aliphatic group may be attached to the ring via a valency bond, a hetero atom or a functional group, such as oxy, carbonyl, carboxyl, sulfonyl, etc.

As examples of cyclic substituents, it is possible to envision cycloaliphatic, aromatic or heterocyclic substituents, in particular cycloaliphatic substituents comprising 6 carbon atoms in the ring or benzene substituents, these cyclic substituents themselves optionally carrying any substituent provided that they do not hinder the reactions involved in the method of the invention. Mention may in particular be made of C₁ to C₄ alkyl or alkoxy groups.

Among the aliphatic groups carrying a cyclic substituent, cycloalkylalkyl groups, for example cyclohexylalkyl groups, or arylalkyl, preferably C₇ to C₁₂ arylalkyl, in particular benzyl or phenylethyl, groups are more particularly targeted.

In general formula (Ia), the groups Rₐ and R_{b} can represent, also independently of one another, a carbocyclic group that is saturated or that comprises 1 or 2 unsaturations in the ring, and that is generally a C₃ to C₈ group, preferably containing 6 carbon atoms in the ring, it being possible for said ring to be substituted. As preferred examples of this type of groups, mention may be made of cyclohexyl groups optionally substituted with linear or branched alkyl groups having from 1 to 4 carbon atoms.

The groups Rₐ and R_{b} can represent, independently of one another, an aromatic hydrocarbon-based group, and in particular benzene group, corresponding to general formula (F₁): 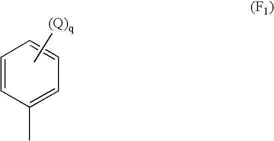
in which:
q represents an integer from 0 to 5,
Q represents a group chosen from a linear or branched C₁ to C₆ alkyl group, a linear or branched C₁ to C₆ alkoxy group, a linear or branched C₁ to C₆ alkylthio group, a group —NO₂, a group —CN, a halogen atom and a group CF₃.

Rₐ and R_{b} can also represent, independently of one another, a polycyclic aromatic hydrocarbon-based group, with it being possible for the rings to form, with one another, ortho-fused or ortho- and peri-fused systems. Mention may more particularly be made of a naphthyl group, it being possible for said ring to be substituted.

Rₐ and R_{b} can also represent, independently of one another, a polycyclic hydrocarbon-based group consisting of at least 2 saturated and/or unsaturated carbocycles or of at least 2 carbocycles, just one of which is aromatic, and which form, with one another, ortho- or ortho- and peri-fused systems. Generally, the rings are C₃ to C₈ rings, preferably C₆ rings. As more particular examples, mention may be made of the bornyl group or the tetrahydronaphthalene group.

As more specific examples of amines corresponding to formula (Ia), mention may be made of aniline, N-methylaniline, diphenylamine, benzylamine and dibenzylamine.

Rₐ and R_{b} can also represent, independently of one another, a saturated, unsaturated or aromatic heterocyclic group containing in particular 5 or 6 atoms in the ring, including one or two hetero atoms such as nitrogen (not substituted with a hydrogen atom), sulfur or oxygen atoms, it also being possible for the carbon atoms of this heterocycle to be substituted.

Rₐ and R_{b} can also represent a polycyclic heterocyclic group defined as being either a group consisting of at least two aromatic or nonaromatic heterocycles containing at least one hetero atom in each ring and forming, with one another, ortho- or ortho- and peri-fused systems, or a group consisting of at least one aromatic or nonaromatic hydrocarbon-based ring and at least one aromatic or nonaromatic heterocycle forming, with one another, ortho- or ortho- and peri-fused systems, it being possible for the carbon atoms of said rings to be optionally substituted.

By way of examples of groups Rₐ and R_{b} of heterocyclic type, mention may be made, inter alia, of furyl, thienyl, isoxazolyl, furazanyl, isothiazolyl, pyridyl, pyridazinyl, pyrimidinyl, pyranyl and phosphino groups, and quinolyl, naphthyridinyl, benzopyranyl and benzofuranyl groups.

Rₐ and R_{b} can be attached so as to constitute, with the carbon atoms which carry them, a monocyclic or polycyclic, saturated, unsaturated or aromatic, heterocyclic group having from 3 to 20 atoms, as defined above. It may comprise two or three ortho-fused rings, which means that at least two rings have two carbon atoms in common. In the case of polycyclic compounds, the number of atoms in each ring preferably ranges between 3 and 6. Rₐ and R_{b} preferably form a ring of pyrimidine or purine type.

The number of substituents present on each ring depends on the carbon number of the ring and on the presence or absence of unsaturations on the ring. The maximum number of substituents that can be carried by a ring is readily determined by those skilled in the art. It is possible for there to be, for example, a C₁ to C₄ alkyl or alkoxy group, an amino group or an oxy (═O) group.

The reagent of the invention can be used to protect the amino groups present in amino acids. As examples of amino acid, mention may be made of glycine, cysteine, aspartic acid, glutamic acid and histidine.

The reagent of the invention is most particularly suitable for protecting relatively non-nucleophilic (deactivated) nitrogen atoms. Thus, it is very advantageous to use it in the synthesis of nucleic acid monomers for protecting the amino groups that are present in the natural bases, such as those derived from pyrimidine (C₄N₂H₄), thymine (C₅N₂O₂H₆) or cytosine (C₄N₃OH₅), and those derived from purine (C₅N₄H₄), adenine (C₅N₅H₅) or guanine (C₅N₅OH₅).

To this effect, reference may be made to that which is described in the state of the art, in particular in the Journal of Organic Chemistry 59, 19, (1994), p. 5767-5773.

According to a practical embodiment, the compound comprising the amino or substituted amino group to be protected can be reacted with the reagent of the invention, in an appropriate solvent.

The solvent is chosen such that it completely or partially solubilizes the reagents and the product obtained.

The molar ratio of the reagent to the compound comprising the group to be protected can greatly vary, for example between 1 and 10, preferably between 1 and 3.

By way of examples of such solvents, mention may be made of nitrites such as acetonitrile or benzonitrile; amides such as dimethylformamide or dimethylacetamide; and aliphatic or aromatic halogenated hydrocarbons, and mention may be made of partially chlorinated hydrocarbons such as dichloromethane or dichloroethane, or aromatic halogenated hydrocarbons such as monochlorobenzene.

The temperature of the reaction is advantageously between 0 and 100° C., preferably between 20 and 60° C.

It should be noted that, if a higher temperature is chosen, the reaction time can be decreased.

The product obtained, comprising the protected group, can be recovered conventionally. It will be specified, for example, that, in the case of the use of the solvent dimethylformamide, at the end of the reaction, water is added and the product formed precipitates such that it can be separated, for example by filtration.

If necessary, the group can be deprotected, for example by means of treatment with a strong acid.

In the present invention, the term “strong acid” denotes an acid having a pKa in water of less than −1.0.

The pKa is defined as the ionic dissociation constant of the acid/base couple, when water is used as solvent.

As preferred examples of strong acids, mention may in particular be made of hydrochloric acid, sulfuric acid, trifluoroacetic acid, methanesulfonic acid and trifluoromethanesulfonic acid.

Use is preferably made of a concentrated acid solution. Commercial solutions, in particular hydrochloric acid (37%), sulfuric acid (95-98%), trifluoroacetic acid, methanesulfonic acid and trifluoromethanesulfonic acid (100%) are more particularly used.

The amount of acid, expressed by the ratio of the number of equivalents of protons to the number of moles of substrate to be deprotected, can range between approximately 2 and 10, preferably between approximately 2 and 5.

The temperature of the deprotection reaction is advantageously between ambient temperature and 60° C.

A deprotection can optionally be carried out under a hydrogen pressure, for example of between 1 and 20 bar, in the presence of a noble metal, preferably deposited on a support. Mention may in particular be made of palladium deposited on carbon black, in a proportion, for example, of between 3 and 5% by weight.

The temperature of the deprotection reaction is in the same temperature range specified above.

Examples of implementation of the invention are given hereinafter. They are given by way of illustration and are not limiting in nature.

### EXAMPLE 1

Preparation of N-(benzyloxycarbonyl)-N′-methylimidazolium triflate

A 1 liter jacketed reactor equipped with a central mechanical stirring mechanism and maintained under an inert nitrogen atmosphere is loaded with 20 g of benzyl chloroformate in 265 g of dichloromethane.

9.5 g of N-methylimidazole are added at ambient temperature (20° C.), with stirring and under an inert atmosphere (nitrogen).

A white suspension then forms in the solvent.

Next, 17.5 g of triflic acid (100%) are added to this suspension.

By the time this addition is complete, the suspension has disappeared, to be replaced with a homogeneous and clear solution.

The solvent is evaporated off under a reduced pressure of 12 mm of mercury.

41.7 g of a white solid are obtained, which corresponds to a yield of 100%.

It can be used as it is without any other purification.

The characteristics of the reagent obtained are as follows:

| melting point: | 77-80° C. |
|---|---|
| ¹H NMR/CDCl₃ (ppm): | |
| H_{(aromatic)}: | 7.4-7.5; |
| H_{(CH2)}: | 5.53; |
| H_{(imidazole)}: | 7.76, 7.50; 9.52; |
| H_{(methyl)}: | 4.05. |

### EXAMPLE 2

Protection of ethyl(adenin-9-yl) acetate

41.5 g of the reagent prepared in example 1 are added, in a single step, to a solution of 10 g of ethyl(adenin-9-yl) acetate in 82 g of anhydrous dimethylformamide under an inert atmosphere and at ambient temperature (20° C.).

The medium is left to stir for 6 hours.

Next, 300 g of water are added to the reaction medium, resulting in precipitation of a pale yellow solid.

This solid is recovered by filtration, washed with water, and then dried under a reduced pressure of 12 mm of mercury.

Thus, 12 g of ethyl-(N⁶-(benzyloxycarbonyl)adenin-9-yl) acetate are obtained, with a purity, determined by high performance liquid chromatography, of greater than 98% (melting point: 140-145° C.).

1-25. (canceled)

26. A method of preparing an acylimidazolium-type reagent of formula (I): 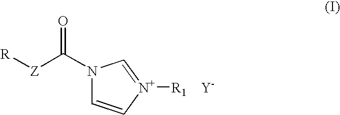
in said formula:
R₁ represents an alkyl or phenyl group,
R represents an alkyl, alkenyl, cycloalkyl, aryl or arylalkyl group,
Z represents a valency bond, an oxygen atom or an NR₂ group; R₂ having the same meaning as R,
Y is an anion originating from an acid whose pKa is less than 1,
wherein said acylimidazolium-type reagent is obtained by reacting:
a reagent comprising a group —COX and corresponding to formula (II): 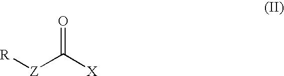
in said formula:
R and Z have the meaning given above,
X represents a bromine or chlorine atom,
and an imidazole reagent of formula (III): 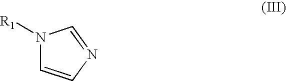
in said formula:
R₁ has the meaning given above,
and then by adding, to the product obtained, a strong acid HY having a pKa of less than 1, which gives the reagent of formula (I), which is recovered.

27. The method as claimed in claim 26, wherein the reagent of formula (II) is of the carboxylic acid chloride or bromide, chloroformate or bromoformate, or carbamoyl chloride or bromide type.

28. The method as claimed in claim 27, wherein the reagent corresponds to formula (II) in which Z represents a valency bond and R represents a linear or branched alkyl group having from 1 to 4 carbon atoms, optionally a methyl or ethyl group.

29. The method as claimed in claim 28, wherein the reagent is acetyl chloride.

30. The method as claimed in claim 27, wherein the reagent corresponds to formula (II) in which Z represents an oxygen atom and R represents a linear or branched alkyl group having from 1 to 4 carbon atoms or a benzyl group.

31. The method as claimed in claim 30, wherein the reagent is an alkyl or benzyl chloroformate or an alkyl or benzyl bromoformate.

32. The method as claimed in claim 27, wherein the reagent corresponds to formula (II) in which Z represents a group NR₂ and in which R and R₂ are identical and represent a linear or branched alkyl group having from 1 to 4 carbon atoms.

33. The method as claimed in claim 32, wherein the reagent is dimethylcarbamoyl chloride.

34. The method as claimed in claim 26, wherein the imidazole reagent corresponds to formula (III) in which the group R₁ represents a linear or branched alkyl group having from 1 to 4 carbon atoms, optionally a methyl group.

35. The method as claimed in claim 34, wherein the preferred reagent is N-methylimidazole.

36. The method as claimed in claim 26, wherein the strong acid is an acid of formula HY in which Y represents the anions BF₄⁻, PF₆⁻, SO₃F⁻ or CF₃SO₃⁻.

37. The method as claimed in claim 36, wherein the strong acid is trifluoromethanesulfonic acid.

38. The method as claimed in claim 26, wherein the reagents are used in an amount such that the ratio of the number of moles of reagent of formula (III) to the number of moles of reagent of formula (II) is chosen between 1 and 1.2, optionally in the region of 1.

39. The method as claimed in claim 26, wherein the acid is added in an amount such that the ratio of the number H⁺ ions to the number of moles of product obtained subsequent to the reaction of reagents (II) and (III) ranges between 0.9 and 1.5, optionally between 1 and 1.1.

40. The method as claimed in claim 26, wherein the reaction is carried out in an organic solvent, optionally a halogenated or nonhalogenated, aliphatic, cycloaliphatic or aromatic hydrocarbon.

41. The method as claimed in claim 40, wherein the solvent is dichloromethane or toluene.

42. The method as claimed in claim 26, wherein the reaction is carried out at a temperature that is between 0° C. and 30° C., optionally at ambient temperature.

43. The method as claimed in claim 26, wherein the reaction is carried out at atmospheric pressure, or under a controlled atmosphere of inert gases.

44. The method as claimed in claim 26, wherein the reaction is carried out with stirring and in dry conditions.

45. The method as claimed in claim 26, wherein the solvent is first introduced, and then the reagent of formula (II), then the reagent of formula (III) is added, optionally gradually, and then the strong acid, preferably gradually, which gives a homogeneous solution comprising the reagent of formula (I).

46. The method as claimed in claim 45, wherein the reagent of formula (I) is recovered in solid form after elimination of the organic solvent.

47. A process for protecting functional groups, optionally amino or substituted amino groups of primary or secondary amines, comprising the step of adding to said amines, a protecting efficient amount of the reagent of formula (I) as defined in claim 26.

48. The process as claimed in claim 47, wherein the functional groups are the amino or substituted amino groups present in nucleic acid bases.

49. The process as claimed in claim 48, wherein the nucleic acid bases are thymine, cytosine, adenine or guanine.
